(19) United States
(12) Patent Application Publication
(10) Pub. No.: US 2014/0311212 A1
(43) Pub. Date: Oct. 23, 2014

(54) GAS SENSOR WITH HEAT SHIELDING

(71) Applicant: Robert Bosch GmbH, Stuttgart (DE)

(72) Inventors: David Boyd, Greenville, SC (US); Craig Magera, Simpsonville, SC (US)

(73) Assignee: Robert Bosch GmbH, Stuttgart (DE)

(21) Appl. No.: 13/947,803

(22) Filed: Jul. 22, 2013

Related U.S. Application Data
(60) Provisional application No. 61/813,922, filed on Apr. 19, 2013.

(51) Int. Cl. G01N 33/00

(52) U.S. Cl. CPC G01N 33/0009
USPC 73/23.2

(57) ABSTRACT

A gas sensor includes a gas sensing element positioned at least partially within a body and exposed at a first end to measure a gas in contact with the first end. The gas sensing element defines an axial direction. A flange extends from the body in a direction transverse to the axial direction. The flange has a first side facing toward the first end and a second side facing toward a remote end of the gas sensor. An O-ring is configured to sealingly position the gas sensor within a bore. An insertion portion of the gas sensor is defined by a wall and configured to hold the O-ring. The wall of the insertion portion is spaced a distance from the body at an axial position of the O-ring to provide a gap therebetween.

### CROSS-REFERENCE TO RELATED APPLICATIONS

This application claims priority to U.S. Provisional Patent Application No. 61/813,922, filed Apr. 19, 2013, the entire contents of which are hereby incorporated by reference herein.

### STATEMENT REGARDING FEDERALLY SPONSORED RESEARCH OR DEVELOPMENT

This invention was made with Government support under DE-EE0005975 awarded by the Department of Energy. The Government has certain rights in this invention.

### BACKGROUND

The present invention relates to various gas (e.g., oxygen) sensor designs. Currently oxygen sensors are designed for high temperature applications. The sensors are mounted in exhaust manifolds or exhaust systems which are inherently designed to handle high temperature due to normal exposure to hot exhaust gas of 1030 C or more. For adaptation to low temperature environments, which may include various plastic or resin components, the sensor itself (e.g., the heated sensing element therein) becomes the primary heat source. At operating temperature, a conventional gas sensor may distort, melt, or otherwise damage adjacent structures not originally intended for such heat.

### SUMMARY

In one aspect, the invention provides a gas sensor having a gas sensing element positioned at least partially within a body and exposed at a first end to measure a gas in contact with the first end. The gas sensing element defines an axial direction. A flange extends from the body in a direction transverse to the axial direction. The flange has a first side facing toward the first end and a second side facing toward a remote end of the gas sensor. An O-ring is configured to sealingly position the gas sensor within a bore. An insertion portion of the gas sensor is defined by a wall and configured to hold the O-ring. The wall of the insertion portion is spaced a distance from the body at an axial position of the O-ring to provide a gap therebetween.

In another aspect, the invention provides a gas sensor having a gas sensing element positioned at least partially within a body and exposed at a first end to measure a gas in contact with the first end. The gas sensing element defines an axial direction. A flange extends from the body in a direction transverse to the axial direction. The flange has a first side facing toward the first end and a second side facing toward a remote end of the gas sensor. An O-ring is configured to sealingly position the gas sensor within a bore. An insertion portion of the gas sensor is defined by a wall and configured to hold the O-ring. There is no heat conduction path radially between the wall of the insertion portion and the body at an axial position of the O-ring.

### BRIEF DESCRIPTION OF THE DRAWINGS

FIG. 1 is a front view of a gas sensor according to one aspect of the invention.

FIG. 2 is a cross-section of the gas sensor of FIG. 1.

FIG. 3 is a front view of a gas sensor according to one aspect of the invention.

FIG. 4 is a cross-section of the gas sensor of FIG. 3.

FIG. 5 is a front view of a gas sensor according to one aspect of the invention.

FIG. 6 is a cross-section of the gas sensor of FIG. 5.

FIG. 7 is a front view of a gas sensor according to one aspect of the invention.

FIG. 8 is a cross-section of the gas sensor of FIG. 7.

FIG. 9 is a schematic thermal model of a gas sensor having no heat shielding.

FIG. 10 is a schematic thermal model of a gas sensor having heat shielding.

### DETAILED DESCRIPTION

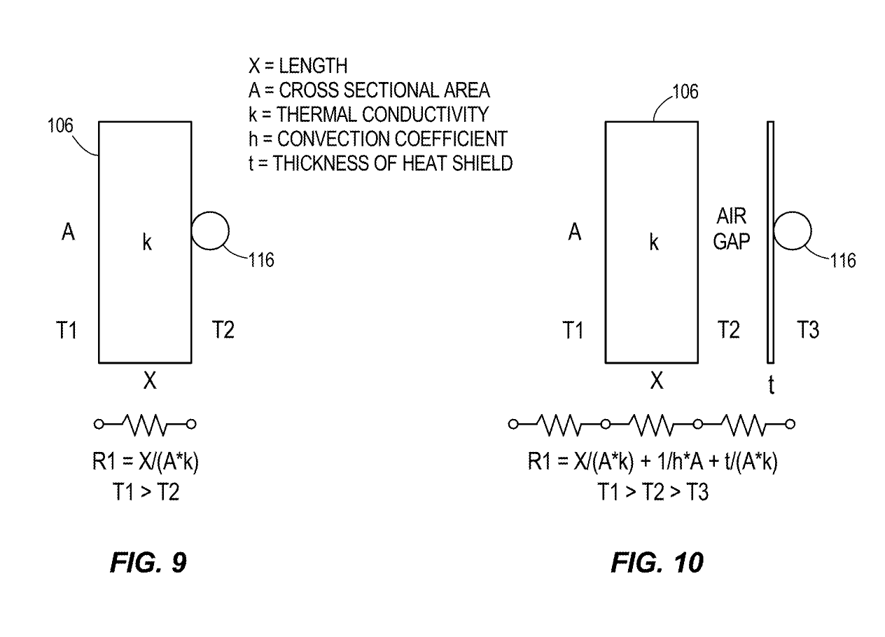

Direct conduction from the metal oxygen sensor housing to the intake manifold or other mounting location is decreased by introducing a gap (e.g., an air gap) and a smaller mass of material in contact with the intake manifold. The heat flow from the housing to the mounting area is by convection and by conduction through a smaller cross section. This reduces the amount of heat transferred to the intake manifold and to the O-ring as shown in the thermal model of FIG. 10 compared to the thermal model of FIG. 9 representing a conventional arrangement. In the various constructions disclosed herein, the mounting surface of the O-ring is displaced from the sensor housing. The temperature rise of the mounting surface is reduced due the gap between the sensor housing and the mounting surface. If the sensor uses an elastomeric O-ring for mounting and sealing, the O-ring is protected from melting or taking a permanent set from the heat.

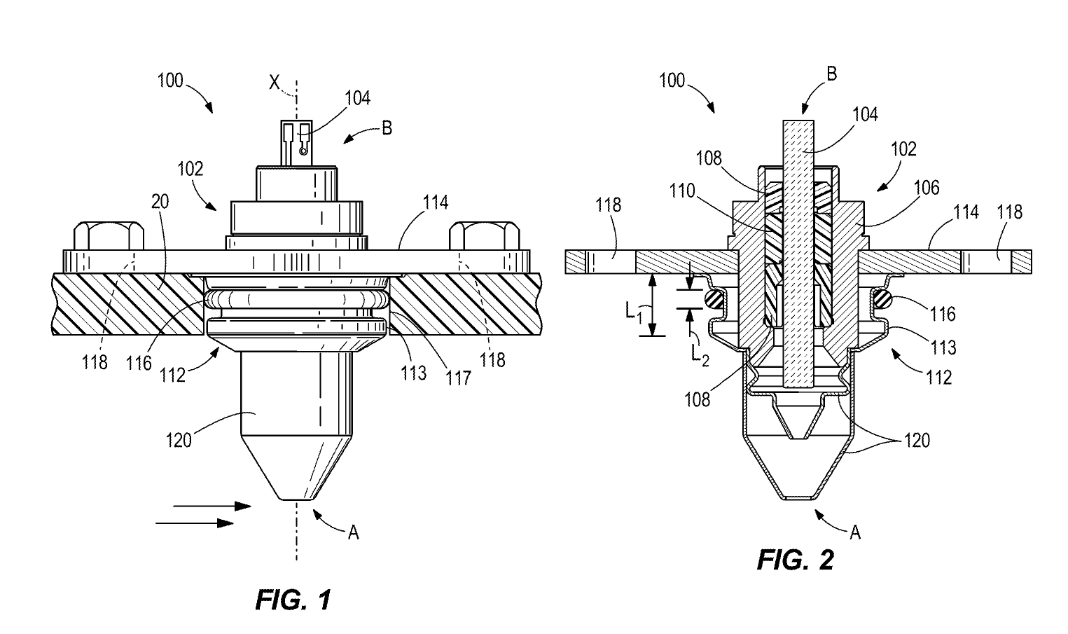

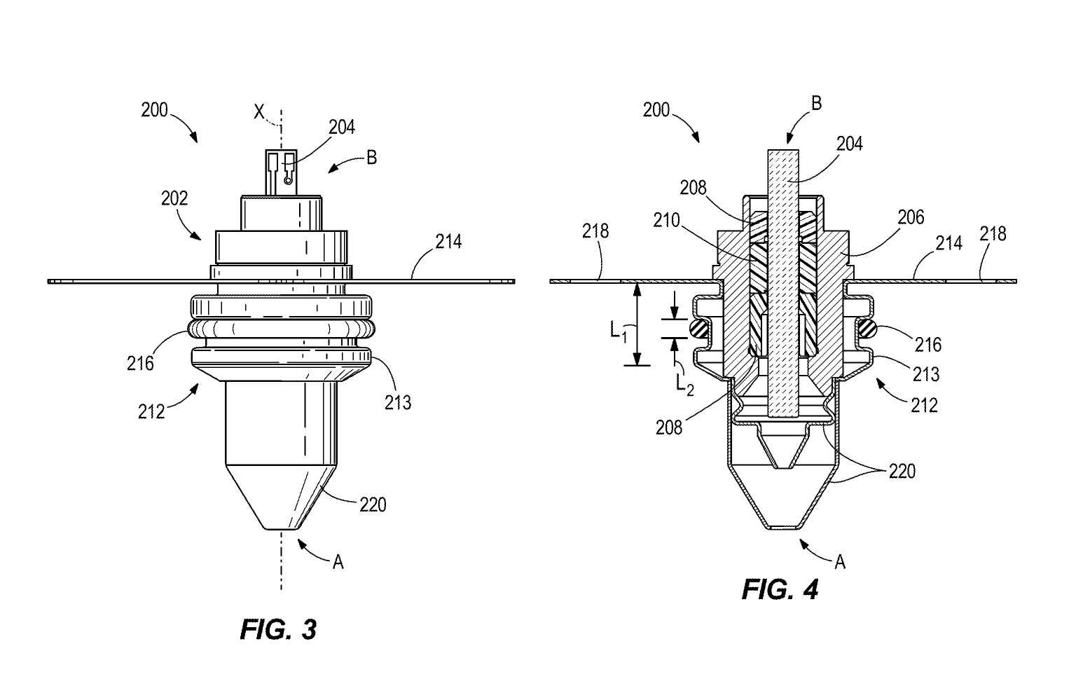

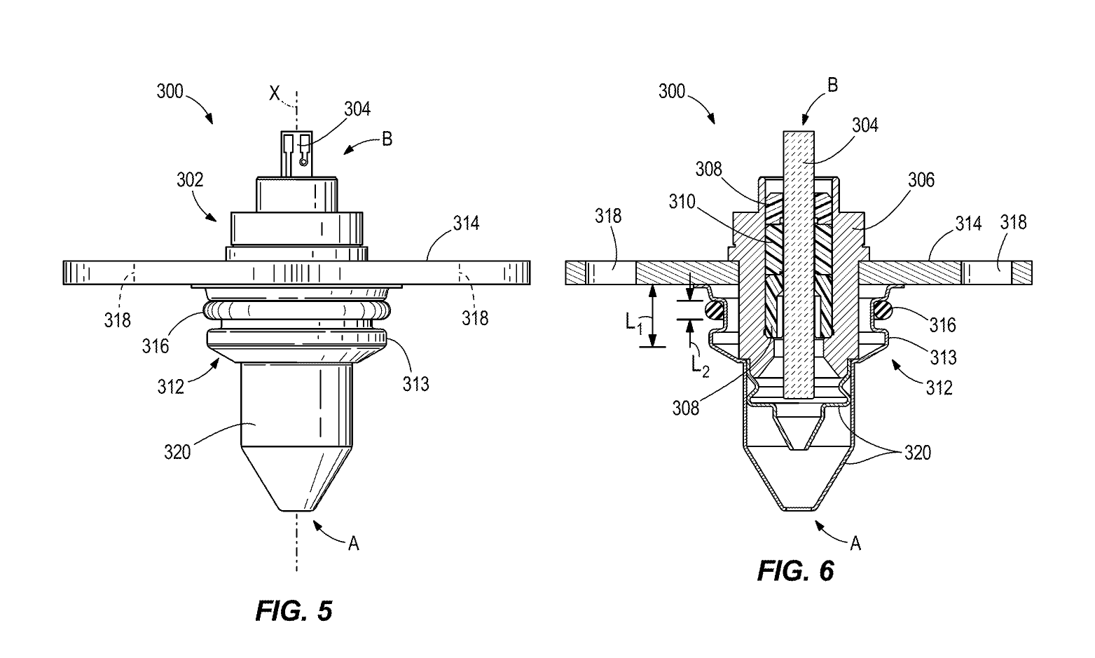

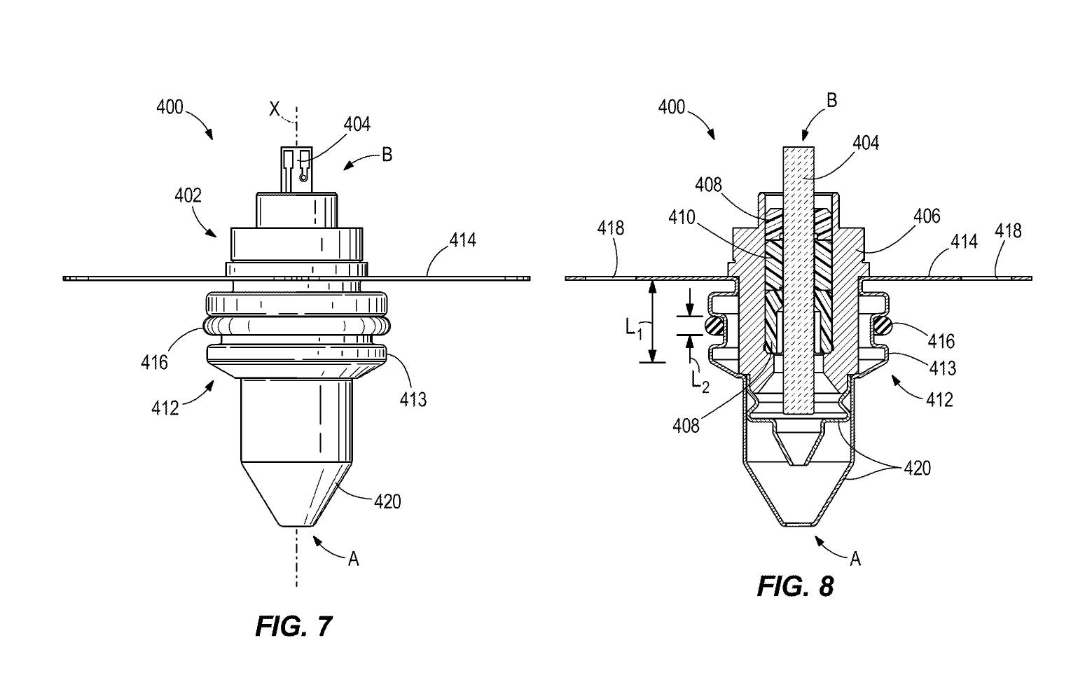

The O-ring heat shield can have several constructions, some of which are described and illustrated herein. FIGS. 1 and 2 illustrate an O-ring heat shield member attached to the sensor at the flange and at the protection tube. FIGS. 3 and 4 illustrate an integrated flange and O-ring heat shield in which an O-ring heat shield member similar to FIGS. 1 and 2 further includes an integrated flange. FIGS. 5 and 6 illustrate an integrated O-ring heat shield and protection tube in which an O-ring heat shield member similar to FIGS. 1 and 2 further includes an integrated outer protection tube. FIGS. 7 and 8 illustrate an integrated flange and O-ring heat shield and protection tube in which an O-ring heat shield member similar to FIGS. 1 and 2 further includes an integrated flange similar to FIGS. 3 and 4, and an integrated outer protection tube similar to FIGS. 5 and 6.

In all cases the device may be made in one piece or as an assembly of pieces from similar or dissimilar materials.

FIGS. 1 and 2 illustrate a gas sensor 100 according to a first construction. The gas sensor 100 is particularly adapted for use in a low temperature (non-exhaust) environment such as an intake manifold 20, for example, of an internal combustion engine. The intake manifold 20 may be non-metallic, and constructed of plastic or resin, for example. In addition, the gas sensor 100 can be used in another portion of an intake system of an internal combustion engine. For example, the gas sensor 100 can be used in a charge air cooler pipe, upstream of a throttle valve and intake manifold and downstream of an intercooler which receives compressed intake gas from a turbocharger. The charge air cooler pipe may also be non-metallic (e.g., plastic or resin).

The gas sensor 100 includes a sensor subassembly (or “short sensor assembly”) 102 that includes a gas sensing element 104 positioned within a sensor sub-housing or body 106 and defining an axis X. The body 106 can be metallic. Ceramic bushings 108 and a soft ceramic seal packing 110 can be used to position the gas sensing element 104 within the body 106. Outside the body 106, an insertion portion 112 and a transverse flange portion 114 are provided. The insertion portion 112 receives an O-ring 116, and is configured to be received within a bore 117 in the intake manifold 20 in sealing relationship. The insertion portion 112 and the O-ring 116 allow the sensor 100 to simply “plug into” the bore 117 in the intake manifold 20 (e.g., simple axial insertion into a non-threaded bore). The flange portion 114 can include one or more apertures 118 to receive fasteners (not shown) for securing the sensor 100 to the intake manifold 20 or other structure. A gasket may also be provided between the flange portion 114 and the intake manifold 20. One or more protection tubes 120 at a first end or sensing end A of the gas sensor 100 cover a sensing end of the sensing element 104, while allowing fluid communication with passing gases. The first end of the sensing element 104 extends from the body 106 and, except for the protection tube(s) 120, is otherwise exposed to ambient gas. When energized, the sensor subassembly 102 enables a gas sensing function of the gas sensor 100 (e.g., an oxygen sensor, such as a pumped-reference wide-band oxygen sensor).

At a second end B of the gas sensor 100 opposite the sensing end A, a connector housing (not shown) may be provided to cover the remote or interior end of the sensing element 104 and provide a plug housing or plug connector portion and electrical terminals or connectors for connection with an external plug member at the remote end B of the gas sensor 100. Alternately, a conventional wire harness can be coupled to the sensing element 104 at the second end B.

It will be noted that the insertion portion 112 is provided by a wall 113 of considerably less thickness than that of the body 106, and furthermore, the wall 113 forming the insertion portion 112 is spaced radially away from the outside of the body 106 to introduce a gap (e.g., an air gap) therebetween. In some constructions, the gap defines a space that is in fluid communication with neither one of a process gas (i.e., gas to be sampled by the sensor 100) nor a reference gas chamber. The wall 113 can be an O-ring heat shield, which is provided to limit the amount of heat transferred from the sensing element 104 to the O-ring 116 during operation of the gas sensor 100. By constructing the gas sensor 100 to limit the heat transfer to the O-ring 116 (and to the insertion portion 112), the materials of the O-ring 116 and the surrounding structure (e.g., intake manifold 20) do not have to be specially modified to accommodate high temperature. For example, the O-ring 116 can be constructed of a common synthetic rubber (e.g., fluoropolymer elastomer such as Viton®), rather than a vastly more expensive perfluoroelastomer O-ring. In some constructions, the wall 113 has a material thickness between about 0.010 inch and about 0.030 inch. In some constructions, the gap between the body 106 and the insertion portion is between about 0.040 inch and about 0.250 inch, measured radially at the axial position of the O-ring 116. The insertion portion 112 can be stamped metal (e.g., steel) in some constructions. The insertion portion 112 may be secured and/or sealed with one or both of the body 106 and the flange portion 114 (e.g., by crimping, laser welding, adhesive bonding, etc.) at its respective ends, but is not in heat conductive relationship with the body 106 at any point between the ends of the insertion portion 112. In other words, the insertion portion 112 has an axial length L₁, between the ends of which, space is maintained between the inside of the wall 113 and any portion of the body 106, the ceramic bushings 108, the seal packing 110, and the sensing element 104. The length L₁ is defined as a portion corresponding to and overlapping with the bore 117 of the manifold 20 in cross-section. The length L₁ of the insertion portion can be at least twice an axial height or length L₂ of the O-ring 116, which is positioned somewhere within the length L₁. FIGS. 9 and 10 are thermal models comparing an O-ring 116 mounted on the body 106 to the O-ring 116 mounted on the insertion portion 112, spaced from the body 106 by the gap.

Although the space between the outside of the body 106 and the inside of the wall 113 may be a closed or sealed space as described above, it may also be a vented space in some constructions. In some constructions, the wall 113 is sealed at a first axial end (e.g., by a circumferentially securing to the transverse flange 114 by laser welding or another means) and unsealed at the opposite second end. Although it may or may not be touching the body 106 at the second end, the second end may be completely free from connection to the body 106. In some constructions, one or more venting apertures are provided in the wall 113.

FIGS. 3 and 4 illustrate a gas sensor 200 according to a second construction. The gas sensor 200 is particularly adapted for use in a low temperature (non-exhaust) environment. Features of the gas sensor 200 that are similar to the gas sensor 100 are not described in detail again, and similar reference numbers are used, incremented by 100.

The gas sensor 200 is identical to the gas sensor 100 of FIGS. 1 and 2, except that the wall 213 of the insertion portion 212 is integrally formed as a single piece with the transverse flange portion 214. For example, the wall 213 of the insertion portion 212 and the transverse flange portion 214 can be stamped as a single contiguous piece. The flange portion 214 can have a wall thickness substantially equal to that of the wall 213. This may be significantly thinner than the thickness of the flange portion 114 of FIGS. 1 and 2, although the flange portion 114 may be provided with a thinner wall thickness in other constructions.

FIGS. 5 and 6 illustrate a gas sensor 300 according to a third construction. The gas sensor 300 is particularly adapted for use in a low temperature (non-exhaust) environment. Features of the gas sensor 300 that are similar to the gas sensors 100, 200 are not described in detail again, and similar reference numbers are used, incremented by 100.

The gas sensor 300 is identical to the gas sensor 100 of FIGS. 1 and 2, except that the wall 313 of the insertion portion 312 is integrally formed as a single piece with a protection tube 320. For example, the wall 313 of the insertion portion 312 and an outer protection tube 320 of a pair of protection tubes 320 can be formed (e.g., stamped) as a single contiguous piece. The protection tube 320 can have a wall thickness substantially equal to that of the wall 313.

FIGS. 7 and 8 illustrate a gas sensor 400 according to a fourth construction. The gas sensor 400 is particularly adapted for use in a low temperature (non-exhaust) environment. Features of the gas sensor 400 that are similar to the gas sensor 100, 200, 300 are not described in detail again, and similar reference numbers are used, incremented by 100.

The gas sensor 400 is identical to the gas sensor 100 of FIGS. 1 and 2, except that the wall 413 of the insertion portion 412 is integrally formed as a single piece with the transverse flange portion 414 as in the gas sensor 200 of FIGS. 3 and 4, and is integrally formed as a single piece with a protection tube 420 as in the gas sensor 300 of FIGS. 5 and 6. For example, the wall 413 of the insertion portion 412, the transverse flange portion 414, and the protection tube 420 (e.g., an outer protection tube) can be stamped as a single contiguous piece. The flange portion 414 and the protection tube 420 can each have a wall thickness substantially equal to that of the wall 413.

Various features and advantages of the invention are set forth in the claims.

What is claimed is:

1. A gas sensor comprising:
a gas sensing element positioned at least partially within a body and being exposed at a first end to measure a gas in contact with the first end, the gas sensing element defining an axial direction;
a flange extending from the body in a direction transverse to the axial direction, the flange having a first side facing toward the first end and a second side facing toward a remote end of the gas sensor;
an O-ring configured to sealingly position the gas sensor within a bore; and
an insertion portion defined by a wall and configured to hold the O-ring, wherein the wall of the insertion portion is spaced a distance from the body at an axial position of the O-ring to provide a gap therebetween.

2. The gas sensor of claim 1, wherein the insertion portion is integrally formed as a single piece with the flange.

3. The gas sensor of claim 1, wherein the insertion portion and the flange are stamped as a single contiguous piece.

4. The gas sensor of claim 1, further comprising a protection tube covering and substantially enclosing the first end of the gas sensing element, wherein the insertion portion is integrally formed as a single piece with the protection tube.

5. The gas sensor of claim 4, wherein the insertion portion and the protection tube are stamped as a single contiguous piece.

6. The gas sensor of claim 1, wherein the O-ring is constructed of a fluoropolymer elastomer.

7. The gas sensor of claim 1, wherein the insertion portion is not in heat conductive relationship with the body at any point between respective axial ends of the insertion portion.

8. The gas sensor of claim 1, wherein the insertion portion has an axial length at least twice an axial length of the O-ring.

9. A gas sensor comprising:
a gas sensing element positioned at least partially within a body and being exposed at a first end to measure a gas in contact with the first end, the gas sensing element defining an axial direction;
a flange extending from the body in a direction transverse to the axial direction, the flange having a first side facing toward the first end and a second side facing toward a remote end of the gas sensor;
an O-ring configured to sealingly position the gas sensor within a bore; and
an insertion portion defined by a wall and configured to hold the O-ring,
wherein there is no heat conduction path radially between the wall of the insertion portion and the body at an axial position of the O-ring.

10. The gas sensor of claim 9, wherein the insertion portion is integrally formed as a single piece with the flange.

11. The gas sensor of claim 9, wherein the insertion portion and the flange are stamped as a single contiguous piece.

12. The gas sensor of claim 9, further comprising a protection tube covering and substantially enclosing the first end of the gas sensing element, wherein the insertion portion is integrally formed as a single piece with the protection tube.

13. The gas sensor of claim 12, wherein the insertion portion and the protection tube are stamped as a single contiguous piece.

14. The gas sensor of claim 9, wherein the O-ring is constructed of a fluoropolymer elastomer.

15. The gas sensor of claim 9, wherein the insertion portion is not in heat conductive relationship with the body at any point between respective axial ends of the insertion portion.

16. The gas sensor of claim 9, wherein the insertion portion has an axial length at least twice an axial length of the O-ring.
